(19) United States
(12) Patent Application Publication
(10) Pub. No.: US 2025/0329430 A1
(43) Pub. Date: Oct. 23, 2025

(54) COMPUTER-IMPLEMENTED SYSTEM AND METHOD FOR VALIDATING PRESCRIPTIONS OF PATIENTS

(71) Applicant: Greenfieldworx LLC, Austin, TX (US)

(72) Inventor: Mark Erwin, Austin, TX (US)

(21) Appl. No.: 18/890,824

(22) Filed: Sep. 20, 2024

Related U.S. Application Data
(60) Provisional application No. 63/637,388, filed on Apr. 23, 2024.

(51) Int. Cl. G16H 20/10; G16H 10/60

(52) U.S. Cl. CPC G16H 20/10; G16H 10/60

(57) ABSTRACT

A computer-implemented system and method for validating prescriptions associated with patients, are disclosed. The computer-implemented method comprises obtaining the prescriptions including information from databases; storing the prescriptions in electronic medical record (EMR) systems; obtaining data comprising at least one of: information associated with the patients whose prescriptions are filled, and metadata of the filled prescriptions, from pharmacies; matching the filled prescriptions obtained from the pharmacies, with the prescriptions stored in the EMR systems; and validating whether each data of the data associated with the filled prescriptions are compliant with a drug affordability and access initiative program, based on the matching of the filled prescriptions obtained from the pharmacies, with the prescriptions stored in the EMR systems and eligibility parameters prestored in the EMR systems.

### CROSS REFERENCE TO RELATED APPLICATION(S)

This application claims the priority to incorporates by reference the entire disclosure of U.S. provisional patent application No. 63/637,388, filed on Apr. 23, 2024, titled “SYSTEM AND METHOD FOR VALIDATING PRESCRIPTION RECONCILIATION PARAMETERS”

### TECHNICAL FIELD

Embodiments of the present disclosure relate to healthcare informatics, and more particularly relate to a computer-implemented system and method for validating one or more prescriptions associated with one or more patients.

### BACKGROUND

Healthcare facilities play a vital role in providing healthcare services to both insured patients and uninsured patients vulnerable to a Human Immunodeficiency Virus (HIV). To support a mission of the healthcare facilities, the healthcare facilities obtain a drug affordability and access initiative certification from a government, thereby allowing the healthcare facilities to earn additional funds for each prescription filled for the insured patients. The additional funds received from pharmacies are crucial for a financial sustainability of the healthcare facilities. However, ensuring a compliance with the drug affordability and access initiative program regulations poses significant challenges. It is essential that the pharmacies accurately report the eligible prescription fills to avoid non-compliance and potential termination of the drug affordability and access initiative program status for the healthcare facilities. Moreover, the healthcare facilities rely heavily on the additional funds, making it imperative to reconcile each prescription filled between pharmacy records and electronic medical records (EMRs) accurately.

Traditional approaches to prescription reconciliation rely on manual data entry and matching, involving cumbersome spreadsheets and time-consuming processes. The healthcare facilities tasked with reconciling prescriptions with the pharmacy records face significant challenges in ensuring an accuracy and the compliance due to the sheer volume of prescriptions processed, the complexity of patient data, and the need to adhere to the stringent drug affordability and access initiative program requirements. The manual reconciliation processes are inherently prone to human error, leading to discrepancies between the pharmacy records and the EMR within healthcare facilities. The errors result in the non-compliance with the drug affordability and access initiative program regulations, potentially leading to financial losses, penalties, and even the termination of the drug affordability.

In the existing technology, a method, and a system of correlating electronic pharmacy data and the EMR are disclosed. The system comprises a correlation module, a first link and a second link to a pharmacy computer network and a second computer network respectively, and a database for storing data from the second computer network. The correlation module, executed by a hardware processor of one or more hardware processors, receives pharmacy queries from the pharmacy computer network and identifies if the pharmacy queries pertain to the EMR in the second computer network. The correlation module converts the pharmacy queries into a protocol of the second computer network and transmits the protocol via the second link. The system also receives electronic medical records (EMR)-based queries from the second computer network, identifies if the EMR-based queries are meant for the pharmacy computer network, converts the EMR-based queries into the protocol of the pharmacy computer network, and transmits the EMR-based queries via the first link. This facilitates seamless communication between the pharmacy data and the EMR. Nevertheless, transmitting the pharmacy queries and the EMR-based queries between the pharmacy computer network and the second computer network via the first link and the second link introduces a communication overhead, potentially leading to delays in query processing and response times.

There are various technical problems with the prescription reconciliation methods in the prior art. In the existing technology, the traditional methods rely on the manual reconciliation processes, requiring a workforce to manually search, match, and reconcile the pharmacy data with the EMR. This is time-consuming and labor-intensive, leading to inefficiencies and increased operational costs. Human involvement in the manual reconciliation processes increases the likelihood of the errors such as data entry mistakes, mismatches, and inconsistencies. The errors lead to inaccuracies in the patient data and financial transactions, compromising patient care and the drug affordability and access initiative program compliance. The manual matching processes are challenging to scale, especially when dealing with large volumes of the pharmacy data and the EMR. As the volume of data increases, a manual workload becomes overwhelming, resulting in bottlenecks and delays in processing. The traditional methods lack robust compliance management mechanisms, making it difficult to ensure adherence to the drug affordability initiative program requirements and regulations.

In addition to the challenges posed by the manual reconciliation process, existing technologies that attempt to automate the prescription reconciliation process fall short of providing comprehensive solutions. Many automated systems lack the sophistication to accurately match prescription data across different datasets, leading to false positives, missed matches, and inefficiencies in the reconciliation workflow. Furthermore, compliance monitoring and reporting within existing systems are limited, leaving the healthcare facilities vulnerable to regulatory scrutiny and audit findings.

Therefore, there is a need for an improved computer-implemented system and method for validating one or more prescriptions associated with one or more patients, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computer-implemented method for validating one or more prescriptions associated with one or more patients is disclosed. The computer-implemented method comprises obtaining, by one or more hardware processors, the one or more prescriptions comprising one or more information, from one or more databases. The one or more prescriptions with the one or more information are generated by one or more healthcare providers and stored in the one or more databases. The one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The computer-implemented method further comprises storing, by the one or more hardware processors, the one or more prescriptions in one or more electronic medical record (EMR) systems.

The computer-implemented method further comprises obtaining, by the one or more hardware processors, one or more data comprising at least one of: information associated with the one or more patients whose prescriptions are filled, and one or more metadata of the one or more filled prescriptions, from one or more pharmacies. The computer-implemented method further comprises matching, by the one or more hardware processors, the one or more filled prescriptions obtained from the one or more pharmacies, with the one or more prescriptions stored in the EMR systems. The computer-implemented method further comprises validating, by the one or more hardware processors, whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more parameters prestored in the EMR systems.

In an embodiment, the computer-implemented method further comprises adapting, by the one or more hardware processors, the one or more pharmacies to generate one or more periodic settlement reports with the one or more data comprising at least one of: the information associated with the one or more patients whose prescriptions are filled, and the one or more metadata of the one or more filled prescriptions.

In another embodiment, the computer-implemented method further comprises storing, by the one or more hardware processors, one or more information associated with at least one of: the one or more healthcare providers, qualified dates of the one or more healthcare providers, the one or more sites at which one or more prescriptions are generated, one or more qualified pharmacies, dates of the one or more qualified pharmacies, name of one or more qualified medicines, and information associated with the one or more filled prescriptions being compliant with the drug affordability and access initiative program.

In yet another embodiment, the computer-implemented method further comprises triggering, by the one or more hardware processors, one or more events indicating that the one or more prescriptions are logged when the one or more information associated with the one or more prescriptions are compared.

In yet another embodiment, the computer-implemented method further comprises (a) generating, by the one or more hardware processors, one or more alerts for one or more issues associated with non-compliance of the one or more prescriptions with the drug affordability and access initiative program; and (b) performing, by the one or more hardware processors, a compliance checking process when the one or more prescriptions are non-compliant with the drug affordability and access initiative program, wherein the compliance checking process comprises reviewing, by the one or more hardware processors, one or more discrepancies caused by one or more non-compliance prescriptions to determine an adherence to requirements and regulations associated with the drug affordability and access initiative program.

In yet another embodiment, the computer-implemented method further comprises performing, by the one or more hardware processors, one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

In yet another embodiment, the computer-implemented method further comprises (a) analyzing, by the one or more hardware processors, one or more financial data to validate the one or more financial data to be compliance with the drug affordability and access initiative program, wherein analyzing the one or more financial data comprises assessing one or more factors comprising at least one of: provider eligibility, program eligibility, and location eligibility; and (b) analyzing, by the one or more hardware processors, the one or more factors to determine whether at least one of: the one or more prescriptions and the one or more financial data are compliant with the drug affordability and access initiative program.

In yet another embodiment, the computer-implemented method further comprises (a) generating, by the one or more hardware processors, a list of issues associated with mappings of the one or more filled prescriptions obtained from the one or more pharmacies, with the one or more prescriptions stored in the EMR systems, for one or more users; (b) adapting, by the one or more hardware processors, the one or more users to map the one or more filled prescriptions obtained from the one or more pharmacies, with the one or more prescriptions stored in the EMR systems, when the one or more filled prescriptions obtained from the one or more pharmacies, are not matched with the one or more prescriptions stored in the EMR systems; (c) learning, by the one or more hardware processors, manually matched mappings of the one or more filled prescriptions obtained from the one or more pharmacies, with the one or more prescriptions stored in the EMR systems; (d) storing, by the one or more hardware processors, information associated with the manually matched mappings of the one or more filled prescriptions obtained from the one or more pharmacies, with the one or more prescriptions stored in the EMR systems, in a memory; and (c) utilizing, by the one or more hardware processors, the stored information associated with the manually matched mappings of the one or more filled prescriptions obtained from the one or more pharmacies, with the one or more prescriptions stored in the EMR systems, from the memory when the one or more filled prescriptions obtained from the one or more pharmacies, with the one or more prescriptions stored in the EMR systems, are not matched.

In one aspect, a computer-implemented system for validating one or more prescriptions associated with one or more patients, is disclosed. The computer-implemented system comprises one or more hardware processors and a memory unit. The memory unit is operatively coupled to the one or more hardware processors. The memory unit comprises a plurality of subsystems in form of programmable instructions executable by the one or more hardware processors. The plurality of subsystems comprises a data obtaining subsystem, a storage subsystem, a prescription matching subsystem, a prescription validating subsystem, and an alerts generating subsystem.

The data obtaining subsystem is configured to obtain the one or more prescriptions comprising one or more information, from one or more databases. The one or more prescriptions with the one or more information are generated by one or more healthcare providers and stored in the one or more databases. The one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The storage subsystem configured to store the one or more prescriptions in one or more electronic medical record (EMR) systems.

The data obtaining subsystem configured to obtain one or more data comprising at least one of: information associated with the one or more patients whose prescriptions are filled, and one or more metadata of the one or more filled prescriptions, from one or more pharmacies.

The prescription matching subsystem configured to match the one or more filled prescriptions obtained from the one or more pharmacies, with the one or more prescriptions stored in the EMR systems.

The prescription validating subsystem configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more parameters prestored in the EMR systems.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 illustrates an exemplary block diagram representation of a network architecture of a computer-implemented system for validating one or more prescriptions associated with one or more patients, in accordance with an embodiment of the present disclosure;

FIG. 2 illustrates a detailed view of the computer-implemented system for validating the one or more prescriptions associated with the one or more patients, such as those shown in FIG. 1, in accordance with an embodiment of the present disclosure;

FIG. 3 illustrates an exemplary flow diagram of the computer-implemented system for validating the one or more prescriptions associated with the one or more patients, in accordance with an embodiment of the present disclosure;

FIG. 4 illustrates an process flow of the computer-implemented system for validating the one or more prescriptions associated with the one or more patients, in accordance with an embodiment of the present disclosure; and

FIG. 5 illustrates a flow chart illustrating a computer-implemented method for validating the one or more prescriptions associated with the one or more patients, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

As used herein the term “drug affordability and access initiative program” is a program that assists the certain healthcare organizations, like hospitals and the healthcare facilities that serve low-income and uninsured patients to buy outpatient drugs at discounted prices. The drug affordability and access initiative program aims to reach more patients and provide the patients with affordable medications. The participating healthcare organizations save money on the medications, allowing the healthcare organizations to better serve their communities in need. Herein in exemplary embodiment, the drug affordability and access initiative program comprises a 340B program.

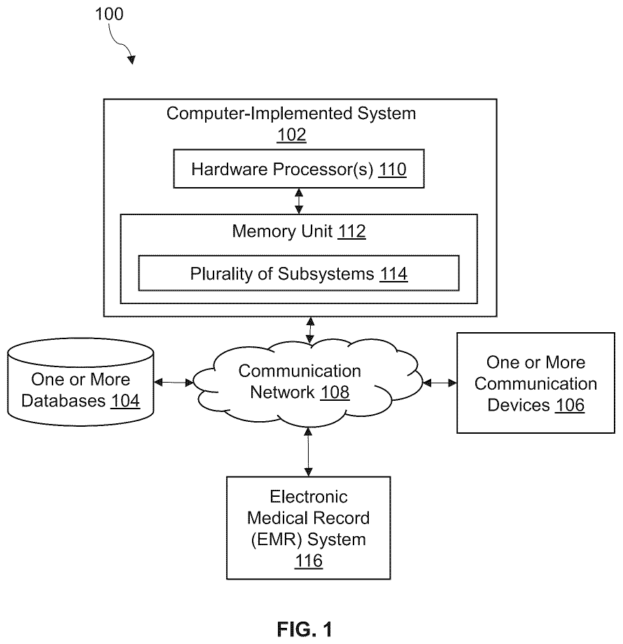

Referring now to the drawings, and more particularly to FIG. 1 through FIG. 5, where similar reference characters denote corresponding features consistently throughout the figures, there are shown preferred embodiments, and these embodiments are described in the context of the following exemplary system and/or method.

FIG. 1 illustrates an exemplary block diagram representation of a network architecture 100 of a computer-implemented system 102 for validating one or more prescriptions associated with one or more patients, in accordance with an embodiment of the present disclosure.

According to an exemplary embodiment of the present disclosure, FIG. 1 depicts the network architecture 100 that may include the computer-implemented system 102, one or more databases 104, and one or more communication devices 106. The computer-implemented system 102 may be communicatively coupled to the one or more databases 104, and the one or more communication devices 106 via a communication network 108. The communication network 108 may be a wired communication network and/or a wireless communication network. The one or more databases 104 may include, but not limited to, storing, managing, and organizing data related to various aspects of an operation of the computer-implemented system 102. The data may include comprise, but not limited to, patient data, medication data, pharmacy data, and other relevant data necessary for a functionality of the computer-implemented system 102. The one or more databases 104 may be any kind of databases including, but not limited to, relational databases, non-relational databases, graph databases, document databases, dedicated databases, dynamic databases, monetized databases, scalable databases, cloud databases, distributed databases, any other databases, and a combination thereof. The one or more databases 104 is configured to support the functionality of the computer-implemented system 102 and enables efficient data retrieval and storage for various aspects associated with the one or more prescription reconciliation parameters.

The integrated network architecture 100 facilitates seamless communication and data exchange, enabling the computer-implemented system 102 to operate cohesively for validating the one or more prescriptions associated with the one or more patients. The capability of the computer-implemented system 102 to validate the one or more prescriptions is underpinned by effective collaboration among the computer-implemented system 102, the one or more databases 104, and the one or more communication devices 106 within the communication network 108.

The computer-implemented system 102 is initially configured to obtain the one or more prescriptions including one or more information, from the one or more databases 104. In an embodiment, the one or more prescriptions with the one or more information are generated and provided by one or more healthcare providers and stored in the one or more databases 104. In an embodiment, the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The computer-implemented system 102 is further configured to store the one or more prescriptions in one or more electronic medical record (EMR) systems 116.

The computer-implemented system 102 is further configured to obtain one or more data including at least one of: information associated with the one or more patients whose prescriptions are filled, and one or more metadata of the one or more filled prescriptions, from one or more pharmacies.

The computer-implemented system 102 is further configured to match the one or more filled prescriptions obtained from the one or more pharmacies, with the one or more prescriptions stored in the EMR systems.

The computer-implemented system 102 is further configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more eligibility parameters prestored in the EMR systems 116.

The computer-implemented system 102 may be hosted on a central server including at least one of: a cloud server or a remote server. In an embodiment, the computer-implemented system 102 may include at least one of: a user device, a server computer, a server computer over the communication network 108, a cloud-based computing system, a cloud-based computing system over the communication network 108, a distributed computing system, and the like. Further, the communication network 108 may be at least one of: a Wireless-Fidelity (Wi-Fi) connection, a hotspot connection, a Bluetooth connection, a local area network (LAN), a wide area network (WAN), any other wireless network, and the like. In an exemplary embodiment, the one or more communication devices 106 may include, but not limited to, a laptop computer, a mobile device, a Smartphone, a Personal Digital Assistant (PDA), a wearable device, a Smart watch, a tablet computer, a phablet computer, and the like.

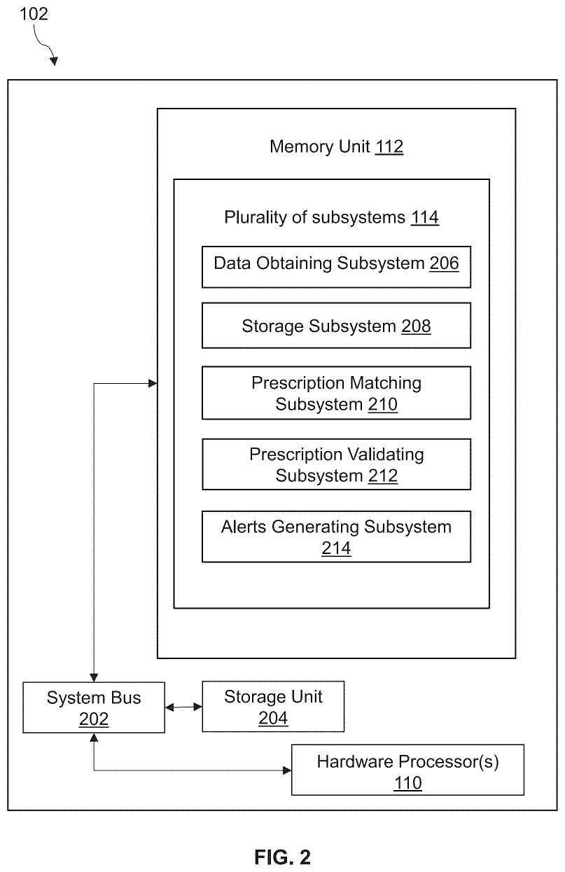

Furthermore, the one or more communication devices 106 may include at least one of: a local browser, a mobile application, and the like. Furthermore, a web application may be used through the local browser and the mobile application to communicate with the computer-implemented system 102. In an embodiment of the present disclosure, the computer-implemented system 102 includes a plurality of subsystems 114. Details on the plurality of subsystems 114 have been elaborated in subsequent paragraphs of the present description with reference to FIG. 2.

Though a few components and subsystems are disclosed in FIG. 1, there may be additional components and subsystems which is not shown, such as, but not limited to, ports, routers, repeaters, firewall devices, network devices, databases, network attached storage devices, servers, assets, machinery, instruments, facility equipment, emergency management devices, image capturing devices, any other devices, and combination thereof. A person skilled in the art should not be limiting the components/subsystems shown in FIG. 1.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the computer-implemented system 102 as is unique to the present disclosure or necessary for an understanding of the present disclosure is depicted and described. The remainder of the construction and operation of the computer-implemented system 102 may conform to any of the various current implementations and practices that were known in the art.

FIG. 2 illustrates a detailed view of the computer-implemented system 102 for validating the one or more prescriptions associated with the one or more patients, such as those shown in FIG. 1, in accordance with an embodiment of the present disclosure.

In an exemplary embodiment, the computer-implemented system 102 comprises the one or more hardware processors 110, the memory unit 112, and a storage unit 204. The one or more hardware processors 110, the memory unit 112, and the storage unit 204 are communicatively coupled through a system bus 202 or any similar mechanism. The memory unit 112 is operatively coupled to the one or more hardware processors 110. The memory unit 112 comprises the plurality of subsystems 114 in the form of programmable instructions executable by the one or more hardware processors 110.

In an exemplary embodiment, the plurality of subsystems 114 comprises a data obtaining subsystem 206, a storage subsystem 208, a prescription matching subsystem 210, a prescription validating subsystem 212, and an alerts generating subsystem 214.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory unit 112 may be a non-transitory volatile memory and a non-volatile memory. The memory unit 112 may be coupled to communicate with the one or more hardware processors 110, such as being a computer-readable storage medium. The one or more hardware processors 110 may execute machine-readable instructions and/or source code stored in the memory unit 112. A variety of machine-readable instructions may be stored in and accessed from the memory unit 112. The memory unit 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 112 includes the plurality of subsystems 114 stored in the form of machine-readable instructions on any of the above-mentioned storage media and may be in communication with and executed by the one or more hardware processors 110.

The storage unit 204 may be a cloud storage or the one or more databases 104 such as those shown in FIG. 1. The storage unit 204 may be any kind of the one or more databases 104 such as, but not limited to, relational databases, dedicated databases, dynamic databases, monetized databases, scalable databases, cloud databases, distributed databases, any other databases, and a combination thereof.

The plurality of subsystems 114 includes the data obtaining subsystem 206 that is communicatively connected to the one or more hardware processors 110. The data obtaining subsystem 206 is configured to obtain the one or more prescriptions including one or more information from one or more databases 104. In an embodiment, the one or more prescriptions with the one or more information are generated by one or more healthcare providers and stored in the one or more databases 104. In an embodiment, the one or more information in the one or more prescriptions may include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The plurality of subsystems 114 includes the storage subsystem 208 that is communicatively connected to the one or more hardware processors 110. The storage subsystem 208 is configured to store the one or more prescriptions in one or more electronic medical record (EMR) systems 116.

The data obtaining subsystem 206 is further configured to obtain the one or more data including at least one of: the information associated with the one or more patients whose prescriptions are filled, and the one or more metadata of the one or more filled prescriptions, from the one or more pharmacies. Prior to obtaining the one or more data from the one or more pharmacies, the one or more pharmacies are configured to fill the one or more prescriptions associated with the one or more patients. The one or more pharmacies are configured to generate one or more periodic settlement reports (e.g., a monthly settlement report) with the one or more data including at least one of: the information associated with the one or more patients whose prescriptions are filled, and the one or more metadata of the one or more filled prescriptions. The one or more pharmacies are configured to share the one or more periodic settlement reports to the computer-implemented system 102.

The plurality of subsystems 114 includes the prescription matching subsystem 210 that is communicatively connected to the one or more hardware processors 110. The prescription matching subsystem 210 is configured to match the one or more filled prescriptions obtained from the one or more pharmacies, with the one or more prescriptions stored in the EMR systems 116. In an embodiment, the computer-implemented system 102 is configured to generate a list of issues associated with mappings of the one or more filled prescriptions obtained from the one or more pharmacies, with the one or more prescriptions stored in the EMR systems, for one or more users and adapt the one or more users to map the one or more filled prescriptions obtained from the one or more pharmacies, with the one or more prescriptions stored in the EMR systems 116, manually, when the one or more filled prescriptions obtained from the one or more pharmacies, are not matched with the one or more prescriptions stored in the EMR systems 116. Further, the prescription matching subsystem 210 is configured to learn manually matched mappings of the one or more filled prescriptions obtained from the one or more pharmacies, with the one or more prescriptions stored in the EMR systems 116. The prescription matching subsystem 210 is further configured to store information associated with the manually matched mappings of the one or more filled prescriptions obtained from the one or more pharmacies, with the one or more prescriptions stored in the EMR systems 116, in a memory (i.e., the one or more databases 104 of the computer-implemented system 102).

The the prescription matching subsystem 210 is further configured to utilize the stored information (i.e., historical information associated with the mappings) associated with the manually matched mappings of the one or more filled prescriptions obtained from the one or more pharmacies, with the one or more prescriptions stored in the EMR systems 116, from the memory when the one or more filled prescriptions obtained from the one or more pharmacies, with the one or more prescriptions stored in the EMR systems 116, are not matched. In other words, the prescription matching subsystem 210 is configured to utilize the historical information associated with the mappings to match the mappings automatically rather than putting in a queue to reduce human work/intervention.

The plurality of subsystems 114 includes the prescription validating subsystem 212 that is communicatively connected to the one or more hardware processors 110. The prescription validating subsystem 212 is configured to validate whether each data of the one or more data associated with the one or more filled prescriptions are compliant with the drug affordability and access initiative program, based on the matching of the one or more filled prescriptions obtained from the one or more pharmacies, with the one or more prescriptions stored in the EMR systems 116 and the one or more eligibility parameters prestored in the EMR systems 116. In an embodiment, the computer-implemented system 102 is configured to generate a list of compliance related issues which are to be determined by the one or more users (e.g., one or more compliance experts). The non-compliance reimbursements are refunded upon determination of the one or more prescriptions are non-compliant to the drug affordability and access initiative program.

The storage subsystem 208 is further configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

The prescription validating subsystem 212 is configured to trigger one or more events indicating that the one or more prescriptions are logged when the one or more information associated with the one or more prescriptions are compared.

The prescription validating subsystem 212 is further configured to analyze one or more financial data to validate the one or more financial data to be compliance with the drug affordability and access initiative program. In an embodiment, analyzing the one or more financial data includes assessing one or more factors including at least one of: provider eligibility, program eligibility, and location eligibility. The prescription validating subsystem 212 is further configured to analyze the one or more factors to determine whether at least one of: the one or more prescriptions and the one or more financial data are compliant with the drug affordability and access initiative program.

The plurality of subsystems 114 further includes the alerts generating subsystem 214 that is communicatively connected to the one or more hardware processors 110. The alerts generating subsystem 214 is configured to generate one or more alerts for one or more issues associated with non-compliance of the one or more prescriptions with the drug affordability and access initiative program. The alerts generating subsystem 214 is further configured to perform a compliance checking process when the one or more prescriptions are non-compliant with the drug affordability and access initiative program. The compliance checking process includes reviewing one or more discrepancies caused by one or more non-compliance prescriptions to determine an adherence to requirements and regulations associated with the drug affordability and access initiative program. Additionally, a compliance queue serves as a designated area where compliance-related tasks are prioritized and managed systematically, ensuring an effective compliance management within the computer-implemented system 102.

In an embodiment, the alerts generating subsystem 214 is configured to perform one or more actions including at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

In an alternative embodiment, one or more cognitive learning models and one or more machine learning models are incorporated into a comparison process that employs one or more algorithms. The one or more algorithms are configured to identify matches by comparing various data points. The one or more algorithms are configured to analyze the one or more prescription reconciliation parameters. Based on the proprietary one or more algorithms, the computer-implemented system 102 is configured to efficiently identify similarities between the one or more pharmacy records and the one or more Electronic Medical Record (EMR) systems 116. In another embodiment, the pharmacy records are imported in the computer-implemented system 102 from the pharmacies in an Excel format.

In an alternative embodiment of the present disclosure, the plurality of subsystems 114 includes the data obtaining subsystem 206 that is communicatively connected to the one or more hardware processors 110. The data obtaining subsystem 206 is configured to serve as a conduit for obtaining one or more patient data, one or more medication data, and one or more pharmacy data from one or more data repository units of the one or more pharmacies and the one or more electronic medical record (EMR) systems 116 associated with the one or more healthcare entities. The one or more patient data, the one or more medication data, and the one or more pharmacy data are obtained through the communication network 108 associated with the one or more communication devices 106. Once obtained, the one or more patient data, the one or more medication data, and the one or more pharmacy data are securely stored within the one or more databases 104 of the computer-implemented system 102, thereby laying a groundwork for subsequent processing and analysis. This seamless obtaining process ensures that the computer-implemented system 102 remains well-equipped with up-to-date and comprehensive data essential for effective healthcare management and decision-making.

In an alternative embodiment of the present disclosure, the plurality of subsystems 114 optionally includes a patient data comparing subsystem that is communicatively connected to the one or more hardware processors 110. The patient data comparing subsystem is configured to employ one or more patient data (i.e., one or more first patient data) from the one or more pharmacy records to establish the accurate match of the one or more patient data with one or more patient data (i.e., one or more second patient data) present in the one or more EMR systems 116 associated with the healthcare facilities. The patient data comparing subsystem employs the one or more cognitive learning models, the one or more machine learning models, and data comparison techniques to ensure precise alignment between the one or more patient data (i.e., the one or more first patient data) obtained from the one or more pharmacy records and the one or more patient data (i.e., the one or more second patient data) stored within the one or more EMR systems 116. The patient data comparing subsystem operates by analyzing various patient attributes, including at least one of: names, dates of birth, addresses, and other demographic details, to identify comparison records within the one or more EMR systems 116.

In other words, the patient data comparing subsystem is configured to determine whether the one or more first patient data associated with the one or more pharmacies are compliant with the one or more second patient data associated with the one or more electronic medical record (EMR) systems 116 within the one or more healthcare entities, by comparing the one or more first patient data associated with the one or more pharmacies with the one or more second patient data associated with the one or more electronic medical record (EMR) systems 116, using at least one of: the one or more cognitive learning models, the one or more machine learning models, and the data comparison techniques.

If the patient data comparing subsystem initially fails to find a match, the patient data comparing subsystem flags the one or more patient data for manual review within a matching queue, where a workforce manually reviews the one or more patient data to confirm the correct patient match, ensuring an accuracy and a consistency in the one or more patient data across the computer-implemented system 102. Once the match is validated, mapping data is created to link the one or more patient data from the one or more pharmacy records to the one or more EMR systems 116 associated with the healthcare facilities. The created mapping data enables the automatic comparison for future records of the same patient from the same pharmacy. Additionally, the established match is retroactively applied to all corresponding records in the matching queue, ensuring the consistency across the one or more patient data and minimizing a manual intervention.

In an alternative embodiment of the present disclosure, the plurality of subsystems 114 optionally includes the medication data comparing subsystem that is communicatively connected to the one or more hardware processors 110. The medication data comparing subsystem is configured to ensure the accurate matching of one or more medication data (i.e., one or more first medication data) between the one or more pharmacy records and one or more medication data (i.e., one or more second medication data) present in the one or more EMR systems 116. In other words, the medication data comparing subsystem is configured to determine whether the one or more first medication data associated with the one or more pharmacies are compliant with the one or more second medication data associated with the one or more electronic medical record (EMR) systems 116 within the one or more healthcare entities, by comparing the one or more first medication data associated with the one or more pharmacies with the one or more second medication data associated with the one or more electronic medical record (EMR) systems 116, using at least one of: the one or more cognitive learning models, the one or more machine learning models, and the data comparison techniques.

Initially, the one or more medication data are compared based on a National Drug Code (NDC), which serves as a unique identifier for each medication. In other words, the one or more first medication data associated with the one or more pharmacies, are compared with the one or more second medication data associated with the one or more electronic medical record (EMR) systems 116, based on information associated with a national drug code (NDC). In an embodiment, the information associated with the national drug code (NDC) is a unique identifier for each medication. However, recognizing that the one or more pharmacies may dispense similar medications that may not precisely match a prescribed drug in the one or more medication data, henceforth the medication data comparing subsystem is configured to incorporate one or more matching criteria (i.e., one or more additional matching criterion).

The one or more matching criteria includes matching medication description, dosage, and quantity to accommodate variations in the medication data dispensed. In other words, the one or more matching criteria including at least one of: the medication description, the dosage, and the quantity, are used to determine similarities between the one or more first medication data associated with the one or more pharmacies and the one or more second medication data associated with the one or more electronic medical record (EMR) systems 116. Furthermore, the medication data comparing subsystem is configured to employ the prior mapping data created during reconciliation efforts or linked NDCs to enhance the matching accuracy. In other words, the medication data comparing subsystem is configured to optimize matching accuracy between the one or more first medication data associated with the one or more pharmacies and the one or more second medication data associated with the one or more electronic medical record (EMR) systems 116, based on at least one of: prior mapped medication data created during a compliance determination process and the national drug code (NDC) linked to each medication.

For instance, a generic medication may be automatically mapped to compare the corresponding brand-name drug, streamlining the comparison process, and reducing the need for the manual intervention. By employing these multiple comparing strategies, the medication data comparing subsystem is configured to ensure the robust and reliable matching of the one or more medication data, thereby enhancing the accuracy and the efficiency of the comparison/matching process.

In an alternative embodiment of the present disclosure, the plurality of subsystems 114 further includes the pharmacy data comparing subsystem that is communicatively connected to the one or more hardware processors 110. The pharmacy data comparing subsystem is configured to ensure the accurate matching of the one or more pharmacy data from the pharmacy records with the one or more pre-stored pharmacy data. In other words, the pharmacy data comparing subsystem is configured to determine whether the one or more pharmacy data are compliant with the one or more pre-stored pharmacy data by comparing the one or more pharmacy data with the one or more pre-stored pharmacy data, using at least one of: the one or more cognitive learning models, the one or more machine learning models, and the data comparison techniques.

The one or more pre-stored pharmacy data are stored within the one or more databases 104 employed for the prescription reconciliation. Additionally, the pharmacy data comparing subsystem is configured to employ other pharmacy information including previously created mapping data to enhance the matching accuracy. Depending on the one or more pharmacy data provided in the pharmacy records, the pharmacy data comparing subsystem determines the most appropriate matching criteria to establish connections between the one or more pharmacy records and the one or more EMR systems 116. By employing these multiple comparing/matching strategies, the pharmacy data comparing subsystem is configured to ensure the robust and reliable matching of the pharmacy data, thereby facilitating the accurate reconciliation and compliance within a drug affordability and access initiative program. In other words, the pharmacy data comparing subsystem is configured to validate the one or more pharmacies in compliant with a drug affordability and access initiative program, based on comparison of the one or more pharmacy data with the one or more pre-stored pharmacy data.

In an alternative embodiment of the present disclosure, the plurality of subsystems 114 further includes the financial information recording subsystem that is communicatively connected to the one or more hardware processors 110. The financial information recording subsystem is configured to compare at least one of: the one or more patient data, the one or more medication data, and the one or more pharmacy data, with one or more prescriptions obtained from the one or more electronic medical record (EMR) systems 116 associated with the one or more healthcare entities. The financial information recording subsystem is further configured to validate accuracy of the one or more prescriptions and one or more financial information based on comparison of at least one of: the one or more patient data, the one or more medication data, and the one or more pharmacy data, with the one or more prescriptions. The financial information recording subsystem is further configured to generate the one or more financial data based on the analysis of the one or more financial information associated with each prescription of the one or more prescriptions.

Moreover, the financial information recording subsystem is configured to maintain the record of expected fills (i.e., one or more records of one or more prescriptions obtained from the one or more electronic medical record (EMR) systems 116), which are reconciled against the actual prescription fills reported by the one or more pharmacies. This proactive approach allows the computer-implemented system 102 to identify and act on any outstanding prescription fills, facilitating timely reconciliation and ensuring a compliance with the drug affordability and access initiative program requirements. The identification and process of one or more identified outstanding prescriptions may assist in accurately recording how much money is involved with each prescription. Additionally, the computer-implemented system 102 keeps track of what prescriptions are expected to be filled and compares the prescriptions to actual occurrences for reconciliation. This way, any missing prescriptions are spotted and addressed promptly, ensuring everything stays in line with the rules of the drug affordability and access initiative program.

In an alternative embodiment of the present disclosure, the plurality of subsystems 114 further includes the compliance determining subsystem that is communicatively connected to the one or more hardware processors 110. The compliance determining subsystem is configured to determine whether at least one of: the one or more prescriptions and the one or more financial data are compliant with the drug affordability and access initiative program upon analysis of the one or more financial information associated with each prescription of the one or more prescriptions. The compliance determining subsystem is configured to ensure adherence to the drug affordability and access initiative program regulations once the prescription fill is accurately recorded through the comparison/matching process. In an embodiment, one or more events are triggered in the computer-implemented system 102 to indicate that the prescription fill is logged when the one or more prescription reconciliation parameters are compared.

In an alternative embodiment of the present disclosure, the plurality of subsystems 114 further includes the report generating subsystem that is communicatively connected to the one or more hardware processors 110. The report generating subsystem is configured to generate one or more reports based on the compliance of at least one of: the one or more prescriptions and the one or more financial data, with the drug affordability and access initiative program. The one or more reports may include, but not limited to, one or more summaries of the one or more prescriptions (i.e., one or more prescription fills), the one or more financial data, one or more compliance statuses of at least one of: the one or more financial data and the one or more prescriptions, and the like.

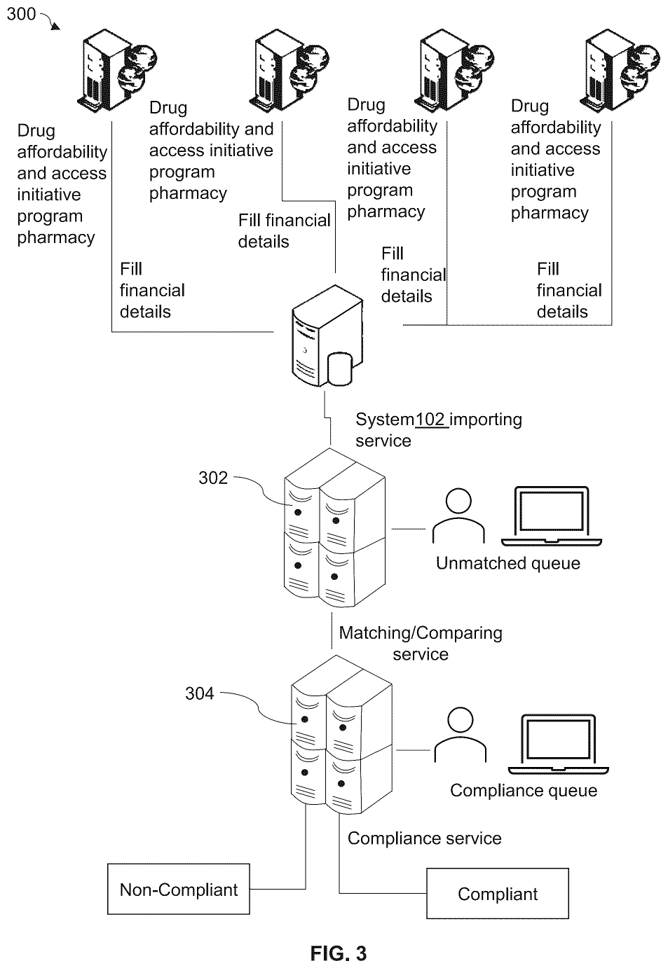

FIG. 3 illustrates an exemplary flow diagram 300 of the computer-implemented system 102 for validating the one or more prescription reconciliation parameters, in accordance with an embodiment of the present disclosure. The computer-implemented system 102 is configured to obtain the one or more data including at least one of: the information associated with the one or more patients whose prescriptions are filled, and the one or more metadata of the one or more filled prescriptions, from one or more pharmacies.

The computer-implemented system 102 is configured to match the one or more filled prescriptions obtained from the one or more pharmacies, with the one or more prescriptions stored in the EMR systems 116. If the one or more filled prescriptions obtained from the one or more pharmacies, are not matched with the one or more prescriptions stored in the EMR systems 116, then a comparison/matching service 302, as shown in FIG. 3, may be configured to adapt the manual review within the matching queue, where the workforce manually reviews for the correct match, across the computer-implemented system 102.

A compliance service 304, as shown in FIG. 3, is configured to determine whether each data of the one or more data associated with the one or more filled prescriptions are compliant with the drug affordability and access initiative program, based on the matching of the one or more filled prescriptions obtained from the one or more pharmacies, with the one or more prescriptions stored in the EMR systems 116 to validate the one or more prescriptions.

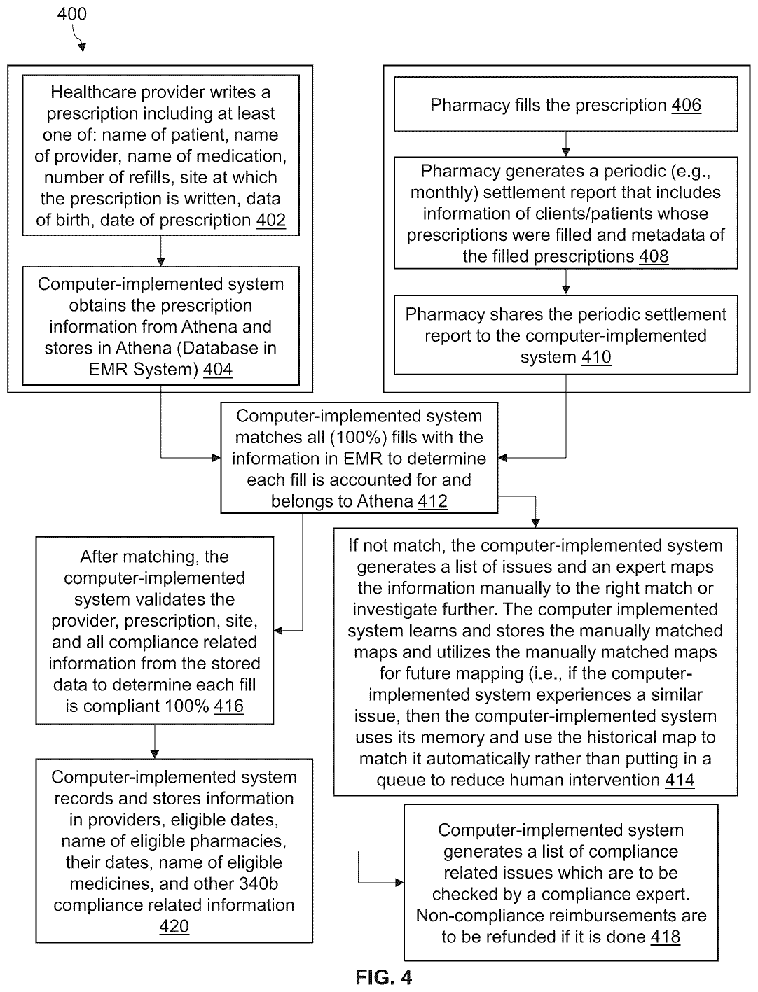

FIG. 4 illustrates an process flow 400 of the computer-implemented system 102 for validating the one or more prescriptions associated with the one or more patients, in accordance with an embodiment of the present disclosure. The one or more healthcare providers generate/write the one or more prescriptions and stores in the one or more databases 104, as shown in step 402. At step 404, the computer-implemented system 102 is configured to obtain the one or more prescription including the one or more information, from one or more databases 104. In an embodiment, the one or more information in the one or more prescriptions include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

At step 406, the one or more pharmacies are configured to fill the one or more prescriptions.

At step 408, the one or more pharmacies are configured to generate the one or more periodic settlement reports (e.g., the monthly settlement report) with the one or more data including at least one of: the information associated with the one or more patients whose prescriptions are filled, and the one or more metadata of the one or more filled prescriptions. At step 410, the one or more pharmacies are configured to share the one or more periodic settlement reports to the computer-implemented system 102.

At step 412, the computer-implemented system 102 is configured to match the one or more filled prescriptions (100% filled prescriptions) obtained from the one or more pharmacies, with the one or more prescriptions stored in the EMR systems 116. At step 414, the computer-implemented system 102 is configured to generate the list of issues associated with mappings of the one or more filled prescriptions obtained from the one or more pharmacies, with the one or more prescriptions stored in the EMR systems, for the one or more users and adapt the one or more users to map the one or more filled prescriptions obtained from the one or more pharmacies, with the one or more prescriptions stored in the EMR systems 116, manually, when the one or more filled prescriptions obtained from the one or more pharmacies, are not matched with the one or more prescriptions stored in the EMR systems 116.

Further, the computer-implemented system 102 is configured to learn manually matched mappings of the one or more filled prescriptions obtained from the one or more pharmacies, with the one or more prescriptions stored in the EMR systems 116. The computer-implemented system 102 is configured to store information associated with the manually matched mappings of the one or more filled prescriptions obtained from the one or more pharmacies, with the one or more prescriptions stored in the EMR systems, in a memory (i.e., the one or more databases 104 of the computer-implemented system 102).

The computer-implemented system 102 is configured to utilize the stored information (i.e., historical information associated with the mappings) associated with the manually matched mappings of the one or more filled prescriptions obtained from the one or more pharmacies, with the one or more prescriptions stored in the EMR systems 116, from the memory when the one or more filled prescriptions obtained from the one or more pharmacies, with the one or more prescriptions stored in the EMR systems 116, are not matched. In other words, the computer-implemented system 102 is configured to utilize the historical information associated with the mappings to match the mappings automatically rather than putting in the queue to reduce the human work/intervention.

At step 416, the computer-implemented system 102 is configured to validate whether each data of the one or more data associated with the one or more filled prescriptions are compliant with the drug affordability and access initiative program, based on the matching of the one or more filled prescriptions obtained from the one or more pharmacies, with the one or more prescriptions stored in the EMR systems 116. At step 418, the computer-implemented system 102 is configured to generate the list of compliance related issues which are to be determined by the one or more users (e.g., one or more compliance experts). The non-compliance reimbursements are refunded upon determination of the one or more prescriptions are non-compliant to the drug affordability and access initiative program.

At step 420, the computer-implemented system 102 is configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

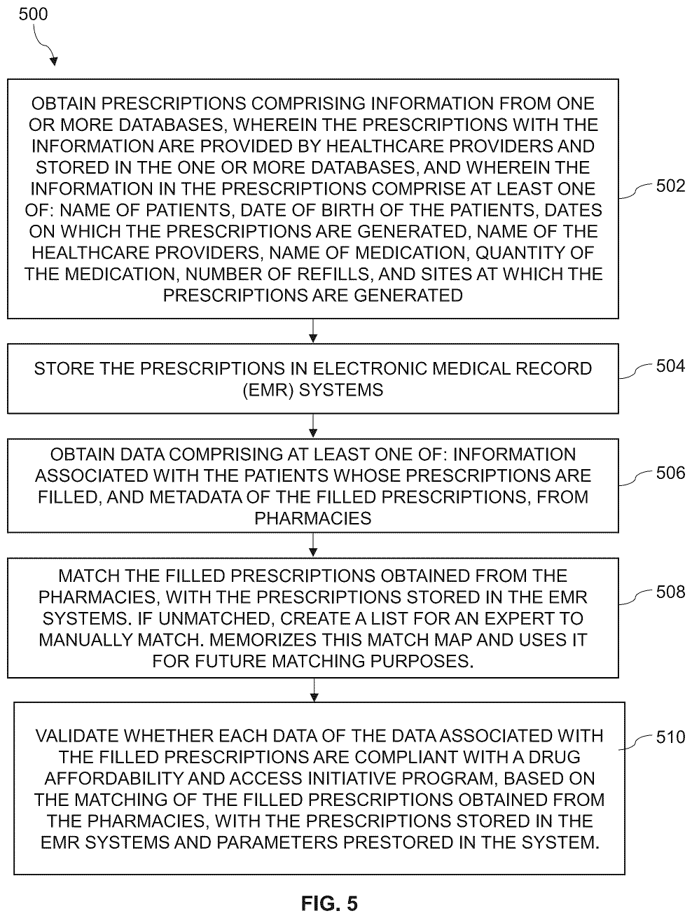

FIG. 5 illustrates a flow chart illustrating a computer-implemented method 500 for validating the one or more prescriptions associated with the one or more patients, in accordance with an embodiment of the present disclosure.

At step 502, the one or more prescriptions include the one or more information, are obtained from the one or more databases 104. The one or more prescriptions with the one or more information are generated by one or more healthcare providers and stored in the one or more databases 104. In an embodiment, the one or more information in the one or more prescriptions may include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

At step 504, the one or more prescriptions are stored in the one or more electronic medical record (EMR) systems 116.

At step 506, the one or more data including at least one of: the information associated with the one or more patients whose prescriptions are filled, and one or more metadata of the one or more filled prescriptions, are obtained from the one or more pharmacies.

At step 508, the one or more filled prescriptions obtained from the one or more pharmacies, are matched with the one or more prescriptions stored in the EMR systems 116.

At step 510, the computer-implemented system 102 validates whether each data of the one or more data associated with the one or more filled prescriptions are compliant with the drug affordability and access initiative program, based on the matching of the one or more filled prescriptions obtained from the one or more pharmacies, with the one or more prescriptions stored in the EMR systems 116 and one or more eligibility parameters prestored in the EMR systems 116.

Numerous advantages of the present disclosure may be apparent from the discussion above. In accordance with the present disclosure, the computer-implemented system 102 for validating the one or more prescriptions is disclosed. By leveraging the capabilities of the computer-implemented system 102 for prescription reconciliation and compliance monitoring, the healthcare facilities/entities ensure accurate recording of the prescription fills and adherence to the drug affordability and access initiative program requirements.

The computer-implemented system 102 assists in maximizing financial benefits derived from the drug affordability and access initiative program and supports a financial sustainability of the participating healthcare facilities. The patients served by the healthcare facilities participating in the drug affordability and access initiative program benefit from reduced out-of-pocket costs for the medications. The discounted prices obtained through the drug affordability and access initiative program make the essential medications more affordable and accessible for the patients, particularly those with limited financial means or without adequate insurance coverage.

The computer-implemented system 102 is configured to effectively correlate the patient data, medication data, and pharmacy data between the pharmacy records and the one or more EMR systems 116. The computer-implemented system 102 is configured to streamline the prescription reconciliation process, minimize manual labor, reduce the errors, ensure compliance with regulatory requirements such as the drug affordability and access initiative program, and provide real-time insights for improved decision-making.

By automating the comparison process, the computer-implemented system 102 significantly reduces the number of human hours required to compare the one or more prescriptions between the one or more pharmacy records and the one or more EMR systems 116. By employing the one or more algorithms for the comparison process, the computer-implemented system 102 minimizes the risk of errors, ensuring that the one or more prescription reconciliation parameters is accurately recorded and reconciled.

By providing a foolproof method for reconciling the prescription fills and validating the compliance with the drug affordability and access initiative program guidelines, the computer-implemented system 102 assists in mitigating the risk of non-compliance and associated financial penalties. This proactive approach to the compliance management safeguards the financial integrity of the healthcare facilities and preserves its eligibility to participate in the drug affordability and access initiative program.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the computer-implemented system 102 either directly or through intervening I/O controllers. Network adapters may also be coupled to the computer-implemented system 102 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer-implemented system 102 in accordance with the embodiments herein. The computer-implemented system 102 herein comprises at least one processor or central processing unit (CPU). The CPUs are interconnected via the system bus 202 to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the computer-implemented system 102. The computer-implemented system 102 can read the inventive instructions on the program storage devices and follow these instructions to execute the methodology of the embodiments herein.

The computer-implemented system 102 further includes a user interface adapter that connects a keyboard, mouse, speaker, microphone, and/or other user interface devices including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that are issued on an application based here on. Accordingly, the embodiments of the present invention are intended to be illustrative, but not limiting, of the scope of the invention, which is set forth in the following claims.

What is claimed is:

1. A computer-implemented method for validating one or more prescriptions associated with one or more patients, the computer-implemented method comprising:
obtaining, by the one or more hardware processors, the one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
storing, by the one or more hardware processors, the one or more prescriptions in one or more electronic medical record (EMR) systems;
obtaining, by the one or more hardware processors, one or more data comprising at least one of: information associated with the one or more patients whose prescriptions are filled, and one or more metadata of the one or more filled prescriptions, from one or more pharmacies;
matching, by the one or more hardware processors, the one or more filled prescriptions obtained from the one or more pharmacies, with the one or more prescriptions stored in the EMR systems;
validating, by the one or more hardware processors, whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more parameters prestored in the EMR systems.

2. The computer-implemented method of claim 1, further comprising:
adapting, by the one or more hardware processors, the one or more pharmacies to generate one or more periodic settlement reports with the one or more data comprising at least one of: the information associated with the one or more patients whose prescriptions are filled, and the one or more metadata of the one or more filled prescriptions.

3. The computer-implemented method of claim 1, further comprising:
storing, by the one or more hardware processors, one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

4. The computer-implemented method of claim 1, further comprising triggering, by the one or more hardware processors, one or more events indicating that the one or more prescriptions are logged when the one or more information associated with the one or more prescriptions are compared.

5. The computer-implemented method of claim 1, further comprising:
generating, by the one or more hardware processors, one or more alerts for one or more issues associated with non-compliance of the one or more prescriptions with the drug affordability and access initiative program; and
performing, by the one or more hardware processors, a compliance checking process when the one or more prescriptions are non-compliant with the drug affordability and access initiative program, wherein the compliance checking process comprises reviewing, by the one or more hardware processors, one or more discrepancies caused by one or more non-compliance prescriptions to determine an adherence to requirements and regulations associated with the drug affordability and access initiative program.

6. The computer-implemented method of claim 5, further comprising performing, by the one or more hardware processors, one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

7. The computer-implemented method of claim 1, further comprising:
analyzing, by the one or more hardware processors, one or more financial data to validate the one or more financial data to be compliance with the drug affordability and access initiative program, wherein analyzing the one or more financial data comprises assessing one or more factors comprising at least one of: provider eligibility, program eligibility, and location eligibility; and
analyzing, by the one or more hardware processors, the one or more factors to determine whether at least one of: the one or more prescriptions and the one or more financial data are compliant with the drug affordability and access initiative program.

8. The computer-implemented method of claim 1, further comprising:
generating, by the one or more hardware processors, a list of issues associated with mappings of the one or more filled prescriptions obtained from the one or more pharmacies, with the one or more prescriptions stored in the EMR systems, for one or more users;
adapting, by the one or more hardware processors, the one or more users to map the one or more filled prescriptions obtained from the one or more pharmacies, with the one or more prescriptions stored in the EMR systems, when the one or more filled prescriptions obtained from the one or more pharmacies, are not matched with the one or more prescriptions stored in the EMR systems;
learning, by the one or more hardware processors, manually matched mappings of the one or more filled prescriptions obtained from the one or more pharmacies, with the one or more prescriptions stored in the EMR systems;
storing, by the one or more hardware processors, information associated with the manually matched mappings of the one or more filled prescriptions obtained from the one or more pharmacies, with the one or more prescriptions stored in the EMR systems, in a memory; and
utilizing, by the one or more hardware processors, the stored information associated with the manually matched mappings of the one or more filled prescriptions obtained from the one or more pharmacies, with the one or more prescriptions stored in the EMR systems, from the memory when the one or more filled prescriptions obtained from the one or more pharmacies, with the one or more prescriptions stored in the EMR systems, are not matched.

9. A computer-implemented system for validating one or more prescriptions associated with one or more patients, the computer-implemented system comprising:
one or more hardware processors;
a memory coupled to the one or more hardware processors, wherein the memory comprises a plurality of subsystems in form of programmable instructions executable by the one or more hardware processors, and wherein the plurality of subsystems comprises:
a data obtaining subsystem configured to obtain the one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
a storage subsystem configured to store the one or more prescriptions in one or more electronic medical record (EMR) systems;
the data obtaining subsystem configured to obtain one or more data comprising at least one of: information associated with the one or more patients whose prescriptions are filled, and one or more metadata of the one or more filled prescriptions, from one or more pharmacies;
a prescription matching subsystem configured to match the one or more filled prescriptions obtained from the one or more pharmacies, with the one or more prescriptions stored in the EMR systems;
a prescription validating subsystem configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more parameters prestored in the EMR systems.

10. The computer-implemented system of claim 9, wherein the one or more pharmacies are configured to generate one or more periodic settlement reports with the one or more data comprising at least one of: the information associated with the one or more patients whose prescriptions are filled, and the one or more metadata of the one or more filled prescriptions.

11. The computer-implemented system of claim 9, wherein the storage subsystem is further configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

12. The computer-implemented system of claim 9, wherein the prescription validating subsystem is further configured to trigger one or more events indicating that the one or more prescriptions are logged when the one or more information associated with the one or more prescriptions are compared.

13. The computer-implemented system of claim 9, further comprising an alerts generating subsystem configured to:
generate one or more alerts for one or more issues associated with non-compliance of the one or more prescriptions with the drug affordability and access initiative program; and
perform a compliance checking process when the one or more prescriptions are non-compliant with the drug affordability and access initiative program, wherein the compliance checking process comprises reviewing one or more discrepancies caused by one or more non-compliance prescriptions to determine an adherence to requirements and regulations associated with the drug affordability and access initiative program.

14. The computer-implemented system of claim 13, wherein the alerts generating subsystem is further configured to perform one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

15. The computer-implemented system of claim 9, wherein the prescription validating subsystem is further configured to:
analyze one or more financial data to validate the one or more financial data to be compliance with the drug affordability and access initiative program, wherein analyzing the one or more financial data comprises assessing one or more factors comprising at least one of: provider eligibility, program eligibility, and location eligibility; and
analyze the one or more factors to determine whether at least one of: the one or more prescriptions and the one or more financial data are compliant with the drug affordability and access initiative program.

16. The computer-implemented system of claim 9, wherein the prescription matching subsystem is further configured to:
generate a list of issues associated with mappings of the one or more filled prescriptions obtained from the one or more pharmacies, with the one or more prescriptions stored in the EMR systems, for one or more users;
adapt the one or more users to map the one or more filled prescriptions obtained from the one or more pharmacies, with the one or more prescriptions stored in the EMR systems, when the one or more filled prescriptions obtained from the one or more pharmacies, are not matched with the one or more prescriptions stored in the EMR systems;
learn manually matched mappings of the one or more filled prescriptions obtained from the one or more pharmacies, with the one or more prescriptions stored in the EMR systems;
store information associated with the manually matched mappings of the one or more filled prescriptions obtained from the one or more pharmacies, with the one or more prescriptions stored in the EMR systems, in a memory; and
utilize the stored information associated with the manually matched mappings of the one or more filled prescriptions obtained from the one or more pharmacies, with the one or more prescriptions stored in the EMR systems, from the memory when the one or more filled prescriptions obtained from the one or more pharmacies, with the one or more prescriptions stored in the EMR systems, are not matched.

17. A non-transitory computer-readable storage medium having instructions stored therein that when executed by one or more hardware processors, cause the one or more hardware processors to execute operations of:
obtaining one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
storing the one or more prescriptions in one or more electronic medical record (EMR) systems;
obtaining one or more data comprising at least one of: information associated with the one or more patients whose prescriptions are filled, and one or more metadata of the one or more filled prescriptions, from one or more pharmacies;
matching the one or more filled prescriptions obtained from the one or more pharmacies, with the one or more prescriptions stored in the EMR systems;
validating whether each data of the one or more data associated with the one or more filled prescriptions are compliant with a drug affordability and access initiative program, based on the matching of the one or more filled prescriptions obtained from the one or more pharmacies, with the one or more prescriptions stored in the EMR systems and one or more parameters prestored in the EMR systems.

18. The non-transitory computer-readable storage medium of claim 17, further comprising adapting the one or more pharmacies to generate one or more periodic settlement reports with the one or more data comprising at least one of: the information associated with the one or more patients whose prescriptions are filled, and the one or more metadata of the one or more filled prescriptions.

19. The non-transitory computer-readable storage medium of claim 17, further comprising storing one or more information associated with at least one of: the one or more healthcare providers, qualified dates of the one or more healthcare providers, the one or more sites at which one or more prescriptions are generated, one or more qualified pharmacies, dates of the one or more qualified pharmacies, name of one or more qualified medicines, and information associated with the one or more filled prescriptions being compliant with the drug affordability and access initiative program.

20. The non-transitory computer-readable storage medium of claim 17, further comprising:
generating one or more alerts for one or more issues associated with non-compliance of the one or more prescriptions with the drug affordability and access initiative program; and
performing a compliance checking process when the one or more prescriptions are non-compliant with the drug affordability and access initiative program, wherein the compliance checking process comprises reviewing one or more discrepancies caused by one or more non-compliance prescriptions to determine an adherence to requirements and regulations associated with the drug affordability and access initiative program.
